(19) United States
(12) Patent Application Publication
(10) Pub. No.: US 2009/0252778 A1
(43) Pub. Date: Oct. 8, 2009

(54) METHODS AND DEVICES FOR REDUCING TISSUE DAMAGE AFTER ISCHEMIC INJURY

(75) Inventors: Theodore L. Parker, Danville, CA (US); Thai Minh Nguyen, Santa Clara, CA (US); John F. Shanley, Emerald Hills, CA (US); Frank Litvack, Los Angeles, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: INNOVATIONAL HOLDINGS LLC., New Brunswick, NJ (US)

(21) Appl. No.: 12/295,879

(22) PCT Filed: Apr. 5, 2007
(86) PCT No.: PCT/US2007/066019

§ 371 Date: Jan. 15, 2009

Related U.S. Application Data
(60) Provisional application No. 60/789,340, filed on Apr. 5, 2006.

(51) Int. Cl. A61F 2/04; A61K 38/28; A61K 31/337; A61K 31/436; A61K 9/00

(52) U.S. Cl. 424/423; 514/4; 514/449; 514/291; 424/400

(57) ABSTRACT

Methods and devices are provided for the delivery of therapeutic agents which reduce myocardial tissue damage due to ischemia and anti-restenotic agents which inhibit restenosis following a cardiac procedure such as stent implantation. The anti-ischemia agents are delivered to the myocardial tissue over an administration period sufficient to achieve reduction in ischemic or reperfusion injury of the myocardial tissue. The anti-restenotic agents are delivered over an administration period sufficient to reduce the re-narrowing of a blood vessel following a cardiac procedure such as implantation of a device. Preferred anti-restenotic drugs are those that do not reduce the beneficial effects provided by the anti-ischemic drug, such as drugs that do not act on the mammalian target of rapamycin (mTOR).

### CROSS REFERENCE TO RELATED APPLICATIONS

This application claims priority to U.S. patent application Ser. No. 11/375,454, filed Mar. 14, 2006 and U.S. Provisional Patent Application Ser. No. 60/662,040, filed Mar. 14, 2005, the entire contents of which are incorporated herein by reference.

### FIELD OF THE INVENTION

This invention is directed to methods and devices for the delivery of therapeutic agents which reduce tissue damage due to ischemia. More particularly, this invention relates to the local delivery of therapeutic agents from implantable medical devices to reduce myocardial tissue damage after ischemic injury.

### BACKGROUND OF THE INVENTION

The reduction or cessation of blood flow to a vascular bed (“ischemia”) accounts for a variety of clinical events that require immediate intervention and restitution of adequate perfusion to the jeopardized organ or tissue. Different tissues can withstand differing degrees of ischemic injury. However, tissues may progress to irreversible injury and cellular necrosis if not reperfused.

Impaired perfusion of cardiac tissue results in a loss of the heart's ability to function properly as the tissue becomes oxygen and energy deprived. Permanent injury is directly related to the duration of the oxygen deficit the myocardium experiences. Ischemia occurs when blood flow to an area of cells is insufficient to support normal metabolic activity. Surgical and percutaneous revascularization techniques following acute myocardial infarction (AMI) are highly effective for treating ischemic myocardial tissue. In the case of an AMI, the main blood flow is stopped by the blockage of a coronary artery and the tissue is perfused only through collateral arteries. Reperfusion is the term used to describe the act of reestablishing blood flow and oxygen supply to ischemic tissue. Reperfusion is essential to the future survival of cells within an ischemic area. Reperfusion may be achieved by a blood flow recanalization therapy, such as coronary angioplasty, administration of a thrombolytic drug, or coronary artery bypass surgery. Timely reperfusion of ischemic myocardium limits infarct size. Early reperfusion with angioplasty or thrombolytic therapy reduces myocardial damage, improves ventricular function, and reduces mortality in patients with AMI. Myocardial salvage can be compromised by such complications as coronary reocclusion and severe residual coronary stenosis.

Reperfusion of the ischemic myocardium does not alone return full functioning of the myocardium. In fact, it is well known that reperfusion itself can cause damage to many cells that survive the initial ischemic event. Studies have shown that reperfusion may accelerate death of irreversibly injured myocardium, and may also compromise survival of jeopardized, but still viable, myocytes salvaged by reperfusion. These so-called reperfusion injuries may represent more than 50% of the ultimate infarct size. A number of cellular mechanisms are believed to be responsible for ischemia-induced reperfusion injury. Development of adjuvant treatments to protect the post-ischemic myocardium and maximize benefits of coronary reperfusion has therefore become a major target of modern cardiovascular research.

Compounds capable of minimizing and containing ischemic or reperfusion damage represent important therapeutic agents. In past years, it has been demonstrated that mortality rates following myocardial infarction and reperfusion can be further reduced by delivery of drugs which optimize energy transfer in post-ischemic heart tissue. For example, an arterial infusion of a combination of glucose, insulin, and potassium (GIK) after an acute myocardial infarction and reperfusion has been shown to provide an impact on injured but viable myocardium tissue and reduce mortality.

The high level of insulin created by the arterial infusion of GIK has been shown to improve ischemic and post-ischemic myocardial systolic and diastolic function as well as improving coronary vasodilatation. The provision of insulin also preserves and restores myocardial glycogen stores. GIK also decreases circulating levels of arterial free fatty acids (FFAs) and myocardial FFA uptake. High FFA levels are toxic to ischemic myocardium and are associated with increased membrane damage, arrhythmias, and decreased cardiac function. Thus, there are many mechanisms by which insulin can reduce ischemic injury. However, when insulin is delivered systemically by arterial infusion, it stimulates glucose and potassium uptake throughout the body and thus reduces glucose and potassium levels in the blood to unsafe levels, resulting in hypoglycemia and hypokolemia. GIK therapy thus involves administration of glucose and potassium along with the insulin to mitigate the undesirable side effects of systemic insulin administration and requires careful monitoring of glucose and potassium levels.

In general, the compounds which have been used for reducing tissue damage after acute myocardial infarction have been delivered systemically, such as by arterial infusion. Systemic delivery of these compounds have significant drawbacks including the requirement for additional administration of protective agents to prevent damage to non-target tissues caused by systemic delivery, i.e. requirement for delivery of glucose and potassium with an insulin infusion. Other drawbacks include the requirement for continuous administration and supervision, suboptimal delivery to the ischemic area, patient discomfort, high dosages required for systemic delivery, and side effects of the systemic delivery and high dosages.

To overcome such problems, local delivery of therapeutic agents for reducing ischemia-induced tissue damage, such as insulin, from a stent or catheter has been described in U.S. Patent Application Publication No. 2004/0142014. Local delivery of therapeutic agents provides the advantage of reduction of ischemic injury, including reduction of reperfusion injury, without the difficulties associated with systemic delivery of the therapeutic agent. U.S. Patent Application Publication No. 2004/0142014 also describes incorporating antirestenotic agents to inhibit restenosis following stent implantation. While this is a beneficial strategy, there is a risk that the anti-restenotic agent will reduce or adversely affect the protection provided by the agents which reduce ischemic injury.

It is therefore an object of the invention to provide methods and devices to reduce tissue damage due to ischemic injury and restenosis by the local administration of anti-ischemic agents and anti-restenotic drugs.

It is a further object of the invention to provide methods and devices for the local administration of a therapeutic agent for reducing ischemic injury and an anti-restenotic drug that does not inhibit the beneficial effects provided by the anti-ischemic drug.

### BRIEF SUMMARY OF THE INVENTION

Methods and devices are provided for the delivery of therapeutic agents which reduce myocardial tissue damage due to ischemia and anti-restenotic agents which inhibit restenosis following a cardiac procedure such as stent implantation. The therapeutic agents are delivered to the myocardial tissue over an administration period sufficient to achieve reduction in ischemic or reperfusion injury of the myocardial tissue. The anti-restenotic drugs are delivered over an administration period sufficient to reduce the re-narrowing of a blood vessel following a cardiac procedure such as implantation of a device. Preferred anti-restenotic drugs are those that do not reduce the beneficial effects provided by the anti-ischemic drug, such as drugs that do not act on the mammalian target of rapamycin (mTOR). Although the agents are preferably delivered together, it is possible to deliver one of the agents systemically, or locally at different times, or both locally and systemically over the same or different periods of time.

In a preferred embodiment, the agents are delivered using an implanted or insertable device releasing an effective amount of one or more anti-ischemic agents and one or more anti-restenotic agents. In one embodiment, a device is implanted at a suitable location in a blood vessel where the device delivers one or more anti-ischemic agents that reduce myocardial tissue damage due to ischemia, such as insulin, and one or more anti-restenotic agents, such as pimecrolimus, that reduce re-narrowing of a blood vessel at the implantation site and downstream of the implantation site over an administration period sufficient to reduce ischemic injury of the surrounding myocardial cells and reduce restenosis. In another preferred embodiment, an occlusion site within a blood vessel is identified; the occlusion treated to achieve reperfusion; and an anti-ischemic agent and anti-restenotic agent locally delivered to the tissue at or near the treated occlusion site and downstream of the occlusion site to reduce ischemic injury and reduce restenosis. In another embodiment, a method for reducing tissue damage following ischemic injury includes identifying an implantation site within a blood vessel; implanting a device containing one or more therapeutic agents that reduce myocardial tissue damage due to ischemia and one or more drugs that inhibit restenosis at the implantation site; and locally delivering the one or more anti-ischemic agents and one or more anti-restenotic drugs from the device to tissue at the implantation site and to the blood vessels downstream of the implantation site over an administration period sufficient to reduce ischemic injury of the surrounding myocardial cells and to reduce or inhibit restenosis.

In alternative embodiments, the anti-ischemic agent or anti-restenotic agent may be delivered systemically in conjunction with local delivery of the anti-restenotic agent or anti-ischemic agent, respectively, from the device.

In another embodiment, a medical device for the local delivery of one or more therapeutic agents that reduce myocardial tissue damage due to ischemia, such as insulin, and one or more anti-restenotic agents to reduce or inhibit restenosis, is implanted. The medical device is configured to be implanted within a coronary artery and one or more of the anti-ischemic agents and/or one or more of the anti-restenotic agents in a biocompatible polymer are affixed to the implantable medical device, wherein therapeutic dosages of the anti-ischemic agent and anti-restenotic agent are released to the myocardial tissue over an administration period effective to reduce ischemic and/or reperfusion injury of the myocardial tissue and to reduce or inhibit restenosis. In a preferred embodiment, the device includes a stent for the local delivery of insulin and one or more anti-restenotic drugs to myocardial tissue, which includes a substantially cylindrical expandable device body configured to be implanted within a blood vessel, and a therapeutic dosage of insulin and one or more anti-restenotic drugs in a biocompatible polymer affixed to the implantable medical device body.

In the methods and devices described above one or more drugs that sensitize tissues to the anti-ischemic agent, such as an insulin sensitizer, may be delivered in conjunction with the anti-ischemic agent and/or anti-restenotic agent, either systemically or locally from the medical device.

### BRIEF DESCRIPTION OF THE DRAWINGS

FIG. 1 is a cross-sectional perspective view of a portion of an expandable medical device implanted in the lumen of an artery with a therapeutic agent arranged for delivery to the lumen of the artery.

FIG. 2 is a perspective view of an expandable medical device showing a plurality of openings.

FIG. 3 is an enlarged side cross-sectional view of a portion of the expandable medical device of FIG. 2.

FIG. 4 is an enlarged side cross-sectional view of an opening illustrating a first therapeutic agent provided for delivery to a lumen of the blood vessel and a second therapeutic agent provided for delivery to a wall of the blood vessel.

FIG. 5 is an enlarged side cross-sectional view of an opening illustrating first and second therapeutic agents for delivery to a lumen of the blood vessel.

FIG. 6a is a graph showing the in vitro cumulative release of insulin over time from a dual drug stent.

FIG. 6b is a graph showing the in vitro cumulative release of pimecrolimus over time from a dual drug stent.

### DETAILED DESCRIPTION OF THE INVENTION

Method and devices are provided for treatment of acute ischemic syndromes including acute myocardial infarction and for reducing injury due to reperfusion of tissue.

### I. DEFINITIONS

First, the following terms, as used herein, shall have the following meanings:

The terms “drug” and “therapeutic agent” are used interchangeably to refer to any therapeutic, prophylactic or diagnostic agent.

The term “anti-ischemic agent” is used to refer to a drug or therapeutic agent that reduces tissue damage due to ischemia and/or reperfusion, or reduces infarct size after AMI.

The term “matrix” refers to a material that can be used to contain or encapsulate a therapeutic, prophylactic or diagnostic agent. As described in more detail below, the matrix may be polymeric, natural or synthetic, hydrophobic, hydrophilic or lipophilic, bioresorbable or non-bioresorbable. The matrix will typically be biocompatible. The matrix typically does not provide any therapeutic responses itself, though the matrix may contain or surround a therapeutic agent, and/or modulate the release of the therapeutic agent into the body. A matrix may also provide support, structural integrity or structural barriers.

The term “biocompatible” refers to a material that, upon implantation in a subject, does not elicit a detrimental response sufficient to result in the rejection of the matrix.

The term “bioresorbable” refers to a matrix, as defined herein, that can be broken down by either a chemical or physical process, upon interaction with a physiological environment, typically into components that are metabolizable or excretable, over a period of time from minutes to years, preferably less than one year.

The term “drug sensitizer” refers to an agent which sensitizes tissue to an anti-ischemic agent, for example, a drug sensitizer can act as an agonist for an agent, can potentiate the activity of an agent, can increase the bioavailability of the agent, or can provide preconditioning or pretreatment which increases the uptake of the agent.

The term “ischemia” refers to a lack of oxygen in a region or tissue. The term typically refers to local hypoxia resulting from obstructed blood flow to an affected tissue.

The term “ischemic injury” as used herein refers to both injury due to obstructed blood flow and reperfusion injury caused by removal of the obstruction and restoration of blood flow.

The term “openings” includes both through openings and recesses.

The term “pharmaceutically acceptable” refers to the characteristic of being non-toxic to a host or patient and suitable for maintaining the stability of a beneficial agent and allowing the delivery of the beneficial agent to target cells or tissue.

The term “polymer” refers to molecules formed from the chemical union of two or more repeating units, called monomers. The term “co-polymer” refers to molecules joined from the chemical union of two or more different monomers. The term “polymer” includes dimers, trimers and oligomers. The polymer may be synthetic, naturally-occurring or semisynthetic. In a preferred form, the term “polymer” refers to molecules which typically have a M_{W} greater than about 3000 and preferably greater than about 10,000 and a M_{W} that is less than about 10 million, preferably less than about a million and more preferably less than about 200,000. Examples of polymers include, but are not limited to, poly-alpha-hydroxy acid esters such as polylactic acid (PLA or DLPLA), polyglycolic acid, polylactic-co-glycolic acid (PLGA), polylactic acid-co-polycaprolactone (PLA/PCL); poly (block-ethylene oxide-block-lactide-co-glycolide) polymers such as (PEO-block-PLGA and PEO-block-PLGA-block-PEO); polyethylene glycol and polyethylene oxide, poly (block-ethylene oxide-block-propylene oxide-block-ethylene oxide); polyvinyl pyrrolidone (PVP); polyorthoesters; polysaccharides and polysaccharide derivatives such as polyhyaluronic acid, poly (glucose), polyalginic acid, chitin, chitosan, chitosan derivatives, cellulose, methyl cellulose, hydroxyethylcellulose, hydroxypropylcellulose, carboxymethylcellulose, cyclodextrins and substituted cyclodextrins, such as beta-cyclo dextrin sulfo butyl ethers; polypeptides and proteins such as polylysine, polyglutamic acid, and albumin; polyanhydrides; polyhydroxy alkanoates such as polyhydroxy valerate and polyhydroxy butyrate.

The term “restenosis” refers to the re-narrowing of an artery following a cardiac procedure such as angioplasty which may include stenosis following stent implantation.

The term “anti-restenotic agent” refers to a compound that can reduce or prevent restenosis as described above.

### II. DRUG DELIVERY DEVICES

Local drug delivery devices, for example, devices in the form of catheters, polymeric delivery devices, and/or stents, can be used to deliver therapeutic agents to ischemic areas, such as myocardial tissue at and downstream of the implantation site when positioned directly at or near a site of a previously occluded blood vessel. The delivery of an anti-ischemic agent locally at the ischemic injury site improves the viability of the cells by reducing ischemic injury to the myocardial cells including reperfusion injury which may occur upon return of blood flow to the ischemic tissue. In cases where reperfusion therapy is performed by angioplasty, a stent is often delivered to the reopened occlusion site. A drug delivery stent for delivery of a therapeutic agent for treatment of ischemic injury and/or anti-restenotic agent can be implanted at the implantation site in the traditional manner after angioplasty. The drug delivery stent for delivery of the therapeutic agent implanted at or near the occlusion site following reperfusion therapy provides the advantage of reduction of ischemic injury including reduction of reperfusion injury without the difficulties associated with systemic delivery of the therapeutic agent. The implantable medical device may also contain one or more drugs that sensitize tissue to the anti-ischemic agent.

Delivery devices can consist of something as simple as a catheter which delivers drug into a blood vessel for release downstream to the affected tissue; polymeric devices which can be in the form of coatings; pellets; particles which contain bioactive molecules that are released by diffusion or degradation of the polymer over time; or a stent. The advantage of the stent is that it can serve the dual purpose of a scaffolding within the blood vessel and release of the bioactive molecules.

Examples of devices for administration of biologically active agent include artificial organs, anatomical reconstruction prostheses, vascular and structural stents, including peripheral stents and coronary stents, vascular grafts and conduits vascular shunts, biological conduits, valve grafts, permanently in-dwelling percutaneous devices, and combinations thereof. Other biomedical devices that are designed to dwell for extended periods of time within a patient that are suitable for the inclusion of therapeutic agents include, for example, Hickman catheters and other percutaneous articles that are designed for use over a plurality of days. Polymeric delivery devices include, for example, U.S. Pat. Nos. 6,491,617 to Ogle, et al., 5,843,156, and 6,290,729 to Slepian, et al. In Slepian, et al., the therapeutic agent is incorporated into a polymeric material which is applied as a thermoplastic coating that is heated to conform to the surface of a vessel, or more preferably, applied in a polymeric material that is in a fluent state at the time of application and photopolymerized in situ.

Examples of methods and materials for application and release of therapeutic agents in a polymeric coating on an implantable medical device are described in U.S. Pat. Nos. 6,273,913 to Wright, et al. and 6,712,845 to Hossainy.

One approach has been to coat a medical device such as a vascular stent with a biologically active agent contained in a polymer matrix, the device may be directly coated with a biologically active agent without a polymer matrix. The compound can be attached using any means that provide a drug-releasing platform. Coating methods include, but are not limited to, dipping, spraying, precipitation, coacervation, vapor deposition, ion beam implantation, and crystallization. The biologically active agent when bound without a polymer can be bound covalently, ionically, or through other molecular interactions including, without limitation, hydrogen bonding and van der Waals forces.

Typically, a coating solution is applied to the device by either spraying a polymer solution onto the medical device or immersing the medical device in a polymer solution. Spraying in a fine spray such as that available from an airbrush will provide a coating with uniformity and will provide control over the amount of coating material to be applied to the medical device. With either a coating applied by spraying or by immersion, multiple application steps can be used to provide improved coating uniformity and improved control. The total thickness of the polymeric coating can range from about 0.1 micron to about 100 microns, preferably between about 1 micron and about 20 microns. The coating may be applied in one coat or, preferably, in multiple coats, allowing each coat to substantially dry before applying the next coat. In one embodiment the biologically active agent is contained within a base coat, and a top coat containing only polymer is applied over the biologically active agent-containing base coat to control release of the biologically active agent into the tissue and to protect the base coat during handling and deployment of the device.

As an alternative to coating an implantable medical device, the therapeutic agent can be deposited within holes, recesses or other macroscopic features within the implantable medical device. Method for depositing a therapeutic agent into holes are described in U.S. Patent Publication No. 2004/0073294 which is incorporated herein by reference in its entirety.

The polymer can be a polymer that is biocompatible and should minimize irritation to the vessel wall when the medical device is implanted. For a stent coating, the polymer should also exhibit high elasticity/ductility, resistance to erosion, and controlled drug release. The polymer may be either a biostable or a bioresorbable polymer depending on the desired rate of release or the desired degree of polymer stability. Bioresorbable polymers that could be used for a coating or within openings include poly(L-lactic acid), polycaprolactone, poly(lactide-co-glycolide), poly(hydroxybutyrate-co-valerate), polydioxanone, polyorthoester, polyanhydride, poly(glycolic acid), poly(D,L-lactic acid), poly(glycolic acid-co-trimethylene carbonate), polyphosphoester, polyphosphoester urethane, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. Biostable polymers with a relatively low chronic tissue response such as polyurethanes, silicones, and polyesters could be used and other polymers could also be used if they can be dissolved and cured or polymerized on the medical device such as polyolefins, polyisobutylene and ethylene-alphaolefin copolymers; acrylic polymers and copolymers, ethylene-co-vinylacetate, polybutylmethacrylate, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile, polyvinyl ketones; polyvinyl aromatics, such as polystyrene, polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers (PEVA); polyamides, such as Nylon® 66 and polycaprolactam; alkyd resins; polycarbonates; polyoxymethylenes; polyimides; polyethers; epoxy resins, polyurethanes; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose.

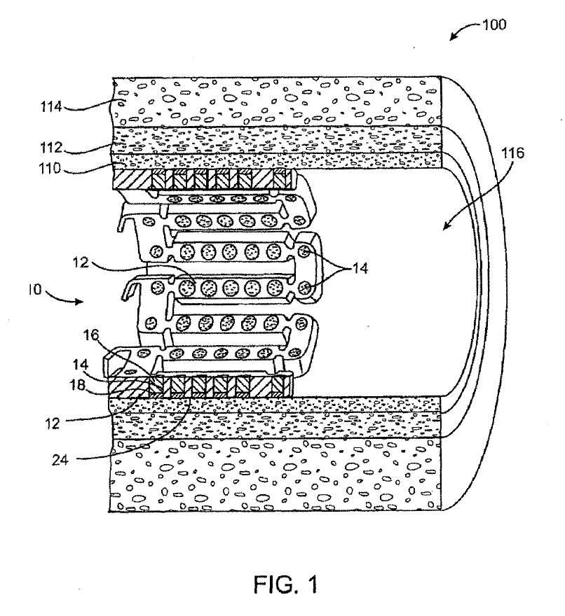

In a preferred embodiment, the device is an expandable stent including polymeric drug delivery reservoirs. FIG. 1 illustrates an expandable medical device 10 in the form of a stent implanted in a lumen 116 of an artery 100. A wall of the artery 100 includes three distinct tissue layers, the intima 110, the media 112, and the adventitia 114. When the expandable medical device 10 is implanted in an artery at an occlusion site, one or more therapeutic agents delivered from the expandable medical device to the lumen 116 of the artery 100 are distributed locally to the tissue at the site of the occlusion and downstream by the blood flow.

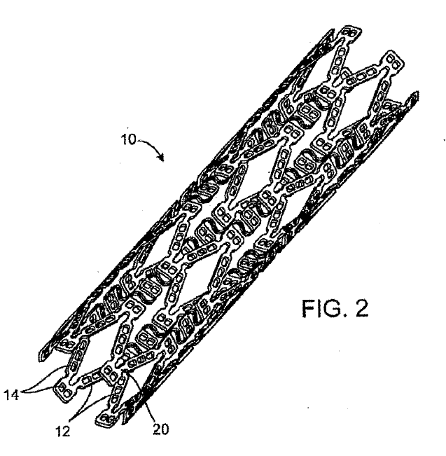

One example of an expandable medical device 10, as shown in FIGS. 1-2, includes large, non-deforming struts 12, which can contain openings 14 without compromising the mechanical properties of the struts, or the device as a whole. The non-deforming struts 12 may be achieved by the use of ductile hinges 20 which are described in detail in U.S. Pat. No. 6,241,762. The openings 14 serve as large, protected reservoirs for delivering various therapeutic agents to the device implantation site and/or downstream of the implantation site.

The relatively large, protected openings 14, as described above, make the expandable medical device particularly suitable for delivering large amounts of therapeutic agents, or genetic or cellular agents, and for directional delivery of agents. The large non-deforming openings 14 in the expandable device 10 form protected areas or reservoirs to facilitate the loading of such agents, and to protect the agent from abrasion, extrusion, or other degradation during delivery and implantation.

FIG. 1 illustrates an expandable medical device for directional delivery of one or more therapeutic agents 16. The openings 14 contain one or more therapeutic agents 16 for delivery to the lumen 116 of the blood vessel and an optional barrier 18 in or adjacent the mural side of the openings. A single opening may contain more than one therapeutic agent or multiple openings may contain only one therapeutic agent. The therapeutic agent in each opening may be the same or different.

The volume of therapeutic agent that can be delivered using openings 14 is about 3 to 10 times greater than the volume of a 5 micron coating covering a stent with the same stent/vessel wall coverage ratio. This much larger therapeutic agent capacity provides several advantages. The larger capacity can be used to deliver multi-drug combinations, each with independent release profiles, for improved efficacy. Also, larger capacity can be used to provide larger quantities of less aggressive drugs and to achieve clinical efficacy without the undesirable side-effects of more potent drugs, such as retarded healing of the endothelial layer.

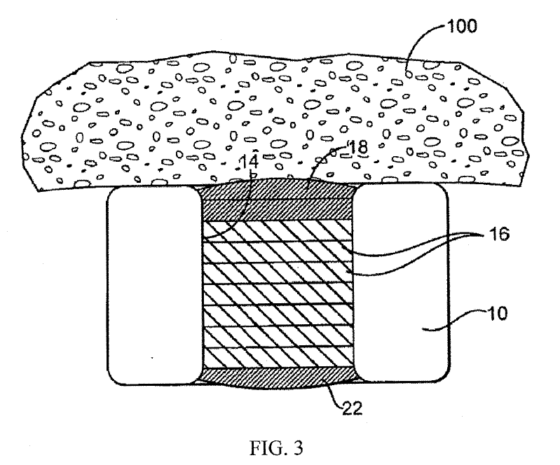

FIG. 3 shows a cross section of a portion of a medical device 10 in which one or more therapeutic agents have been loaded into an opening 14 in multiple deposits. Although multiple discrete layers are shown for ease of illustration, the layers may be discrete layers with independent compositions or blended to form a continuous polymer matrix and agent inlay. For example, the layers can be deposited separately in layers of a drug, polymer, solvent composition which are then blended together in the openings by the action of the solvent. The agent may be distributed within an inlay uniformly or in a concentration gradient. Examples of some methods of creating such deposits and arrangements of layers are described in U.S. Patent Publication No. 2002/0082680, which is incorporated herein by reference in its entirety. The use of drugs in combination with polymers within the openings 14 allows the medical device 10 to be designed with drug release kinetics tailored to the specific drug delivery profile desired.

According to one embodiment, the openings have an area of at least 5×10⁻⁶ square inches, and preferably at least 10×10⁻⁶ square inches.

In the example of FIG. 3, the mural side of the openings are provided with a cap region 18 which is a region of polymer or other material having an erosion rate which is sufficiently slow to allow substantially all of the therapeutic agent in the therapeutic agent region 16 to be delivered from the luminal side of the opening prior to erosion of the cap region. The cap region 18 prevents loss of the therapeutic agent during transport, storage, and during the stent implantation procedure. However, the cap region 18 may be omitted where mural and luminal delivery of the agent is acceptable.

In one example, the cap region 18 and/or a base region 22 may be formed by a material soluble in a different solvent from the therapeutic agent region 16 to prevent intermixing of regions during fabrication. For example, where one or more deposits of therapeutic agent and matrix have been deposited in the openings in a solvent (e.g. Insulin and PVP in water), it may be desirable to select a different polymer and solvent combination (e.g. PLGA in anisole) for the cap region to prevent the therapeutic agent from mixing into the cap region. In addition to the cap 18 and base 22, other therapeutic agent regions, protective or separating regions may also be formed of non-mixing polymer/solvent systems in this manner.

The base 22 can provide a seal during filling of the openings. The base 22 is preferably a rapidly degrading biocompatible material when providing luminal delivery.

Since the cap region 18 and therapeutic agent 16 are created independently, individual chemical compositions and pharmacokinetic properties can be imparted to each layer. Numerous useful arrangements of such layers can be formed, some of which will be described below. Each of the layers may include one or more agents in the same or different proportions from layer to layer. Changes in the agent concentration between layers can be used to achieve a desired delivery profile. For example, a decreasing release of drug for about 24 hours can be achieved. In another example, an initial burst followed by a constant release for about one week can be achieved. Substantially constant release rates over time period from a few hours to months can be achieved. The layers may be solid, porous, or filled with other drugs or excipients.

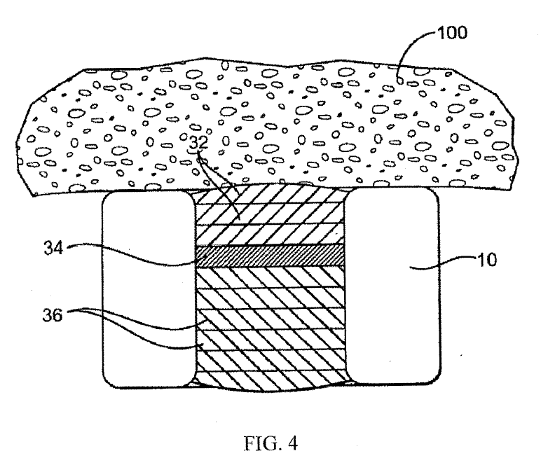

FIG. 4 is a cross sectional view of a portion of an expandable medical device 10 including two or more therapeutic agents including an anti-ischemic agent and an anti-restenotic agent. Dual agent delivery systems such as that shown in FIG. 4 can deliver two or more therapeutic agents in different directions for the treatment of different conditions or stages of conditions. For example, a dual agent delivery system may deliver a drug for treatment of ischemia 36 luminally and an anti-restenotic agent 32 murally from the same or different openings in the same drug delivery device.

A third therapeutic agent, for example, a sensitizing agent, can also be provided at the mural side of the device 10 in one or more layers in addition to the therapeutic agent 36 and the anti-restenotic agent 32 for reducing ischemic injury. Optionally, a separating layer 34 can be provided between the agent layers. A separating layer 34 can be particularly useful when the administration periods for the two agents are substantially different and delivery of one of the agents will be completed while the other agent continues to be delivered. The separating layer 34 can be any biocompatible material, which is preferably biodegradable at a rate which is equal to or longer than the longer of the administration periods of the two agents. The device of FIG. 4 is illustrated without a base 22, however, the base of FIG. 3 can be used if needed.

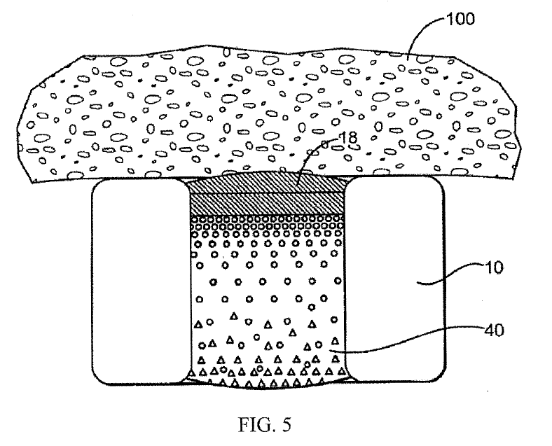

FIG. 5 illustrates an expandable medical device 10 including an inlay 40 formed of a biocompatible matrix with first and second agents provided in the matrix for delivery according to different agent delivery profiles. As shown in FIG. 5, a first drug illustrated by triangles (such as an anti-ischemic agent) is provided in the matrix with a concentration gradient such that the concentration of the drug is highest adjacent the luminal side of the opening and is lowest at the mural side of the opening. The second drug, illustrated by circles, is relatively concentrated in an area close to the mural cap region 18 in the opening. This configuration illustrated in FIG. 5 results in delivery of two different agents with different delivery profiles and in different primary directions from the same inlay 40. In addition to, or as an alternative to the two agents provided in the matrix 40, one or more agents can be added to the cap region 18 or to a base region (not shown). For example, a drug sensitizer can be added to the base region of the embodiment of FIG. 5.

In the embodiments described above, the therapeutic agent can be provided in the expandable medical device in a biocompatible matrix. The matrix can be bioresorbable or can be a permanent part of the device from which the therapeutic agent diffuses. One or more barrier regions, separating regions, and cap regions can be used to separate therapeutic agents within the openings or to prevent the therapeutic agents from degradation or delivery prior to implantation of the medical device.

In an exemplary embodiment, the stent is loaded with three regions, a base, a drug, and a cap. The base is a bioresorbable polymer, such as PLGA 85:15. The base can also be formed of a non-bioresorbable polymer, or a mixture of bioresorbable and non-bioresorbable polymers. The therapeutic agent, for example, insulin, is provided in a combination of a polysaccharide such as trehalose and a bioresorbable polymer such as polyvinyl pyrollidone (“PVP”). The cap is one or more slow degrading polymers, such as PLA/PCL copolymer and/or PLGA 50:50. The cap is deposited in a solvent which does not dissolve the constituents of the underlying drug region, for example, for the drug insulin the cap can be deposited in anisole.

The drug sensitizer, for example, an insulin sensitizer, can be combined with a biodegradable polymer, such as PLGA or PVP and standard solvents including DMSO, NMP, water, and combinations of these. The therapeutic agent for reducing ischemic injury and drug sensitizer may be loaded in the same reservoir or different reservoirs. When the drugs are loaded in the same reservoir, the drugs can be separated by a separating layer (not shown) or mixed together in a matrix as shown in FIG. 5. Approximately, up to about 500 μg of therapeutic agent may be loaded in the reservoirs of a standard coronary stent having a length of about 16 mm. Other amounts may be loaded in reservoirs of other devices. In a preferred embodiment, about 100-300 μg of insulin are loaded in the reservoirs of a standard 16 mm coronary stent.

In another example, insulin and/or the insulin sensitizer can be combined with a hydrogel or proto-hydrogel matrix. The insulin and/or insulin sensitizer/hydrogel is loaded into the openings of a stent and dehydrated. Rehydration of the hydrogel causes the hydrogel to swell and allows the insulin and/or insulin sensitizer to be released from the hydrogel.

### III. DRUGS INCORPORATED INTO THE MEDICAL DEVICES FOR REDUCING ISCHEMIC INJURY AND RESTENOSIS

In one embodiment, a stent or other local delivery device may be used for local delivery of one or more therapeutic agents following acute myocardial infarction and reperfusion. In preferred embodiments, the stent or another local delivery device is used for the delivery of an anti-ischemic agent which reduces myocardial tissue damage due to ischemia, such as insulin, and one or more anti-restenotic drugs, such as pimecrolimus or paclitaxel, which reduces or inhibits restenosis. Preferably, the anti-restenotic drug is one that does not reduce or adversely affect the beneficial effects provided by the anti-ischemic agent. Optionally, the stent or local delivery device may contain a drug sensitizer that sensitizes target (myocardial) tissue to the therapeutic agent, such as an insulin sensitizer.

A. Anti-Ischemic Agents

Insulin is a hormone which improves glycolic metabolism and ATP production. Insulin also may act as a vasodilator, an anti-inflammatory, and an antiplatelet agent. Thus, insulin acts by several mechanisms to decrease infarct size by reducing inflammation, slowing the rate of ischemic necrosis, decreasing circulating levels of FFA and myocardial FFA uptake, restoring myocardial glycogen stores and improving contractile function. The insulin can be human, non-human, or synthetic and can be complete or fragments. Preferably the insulin is a stable, short acting form which is resistant to radiation. Insulin in its crystalline form may be used for improved resistance to radiation. When the insulin is combined with a polymer an agent may be added to preserve bioactivity. Insulin has been found to retain its bioactivity for administration periods of at least 24 hours when delivered in poly(lactide-co-glycolide) (PLGA). For substantially longer administration periods, an antacid or other agent may be used to maintain a required pH for continued bioactivity from a PLGA matrix.

Other drugs which are particularly well suited for the reduction of ischemic injury following acute myocardial infarction or other ischemic injuries include, but are not limited to, vasodilators such as adenosine, dipyridamole and cilostazol; nitric oxide donors; prostaglandins and their derivatives; antioxidants including hydroxyflavonols and dihydroxy; membrane stabilizing agents; anti-TNF compounds; anti-inflammatories including dexamethasone, aspirin, pirfenidone, meclofenamic acid, and tranilast; hypertension drugs including Beta blockers, ACE inhibitors, and calcium channel blockers; anti-metabolites such as 2-CdA; vasoactive substances including vasoactive intestinal polypeptides (VIP); insulin; protein kinases; antisense oligonucleotides including resten-NG; immunosuppressants including sirolimus, everolimus, tacrolimus, etoposide, cyclosporins such as cyclosporine A and mitoxantrone; antithrombins; antiplatelet agents including tirofiban, eptifibatide, and abciximab; cardio protectants including pituitary adenylate cyclase-activating peptide (PACAP), apoA-I milano, amlodipine, nicorandil, cilostaxone, and thienopyridine; anti-leukocytes; cyclooxygenase inhibitors including COX-1 and COX-2 inhibitors; petidose inhibitors which increase glycolitic metabolism including omnipatrilat; calcium sensitizers including lerosimendan, semidan and pimobendan.

Protein or peptide drugs can be human, non-human, recombinant or synthetic and can be the full length native form or an active fragment thereof. Preferably the insulin is a stable, short acting form which is resistant to radiation. Insulin in its crystalline form may be used for improved resistance to radiation. When the insulin is combined with a polymer, an agent may be added to preserve bioactivity. Insulin has been found to retain its bioactivity for periods of at least 24 hours when delivered in poly(lactide-co-glycolide) (PLGA). For substantially longer administration periods, a buffering agent such as hydroxyapatite may be used to maintain the pH as the polymer degrades to release acidic byproducts.

Agents for the treatment of ischemic injury may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods. Mechanical methods include direct DNA microinjection, ballistic DNA-particle delivery, liposome-mediated transfection, and receptor-mediated gene transfer. Vector-mediated delivery typically involves recombinant virus genomes, including but not limited to those of retroviruses, adenoviruses, adeno-associated viruses, herpesviruses, vaccinia viruses, picornaviruses, alphaviruses, and papovaviruses.

B. Anti-Restenotic Drugs

In another embodiment, one or more anti-restenotic drugs are delivered primarily from a mural side of a stent to inhibit restenosis, in addition to the agent or agents delivered primarily from the luminal side of the stent for reduction of ischemic injury. The primarily murally delivered agents may include antineoplastics, anti-mitotics, anti-inflammatories, antiangiogenics, angiogenic factors, anti-thrombotics, such as heparin, antiproliferatives, such as paclitaxel and Pimecrolimus and derivatives thereof.

In a preferred embodiment, the anti-restenotic drug is one that does not reduce or adversely affect the protection provided by the anti-ischemic agents. Examples of such anti-restenotic drugs include those that do not act on the mammalian target of rapamycin (mTOR), such as Pimecrolimus, tacrolimus and paclitaxel. Assays for testing whether drugs act on mTOR can be performed as described in Brunn et al. EMBO J. 15(19):5256-67 (1996); Sabers et al. J. Biol. Chem. 270(2):815-22 (1995); and Abraham, R T and Wiederrecht, G J. Annu Rev Immunol. 14:483-510 (1996). Among the anti-restenotic agents which act on mTOR and inhibit the beneficial effects of insulin are rapamycin.

C. Drug Sensitizers

Insulin sensitizers, such as biguanides, thiazolidinediones, and glitazars can be used in combination with insulin to enhance the effect of insulin. The insulin sensitizers can be incorporated into a stent or other local delivery device along with insulin for local delivery, or one of the drugs can be administered systemically at the same time or shortly before or after the other drug is administered locally from a stent or other local delivery device.

The biguanides that can be used include metformin and phenformin. These compounds have been well described in the art, e.g. in U.S. Pat. No. 6,693,094. Metformin (N,N-dimethylimidodicarbonimidicdiamide; 1,1-dimethylbiguanide; N,N-dimethylbiguanide; N,N-dimethyldiguanide; N′-dimethylguanylguanidine) is an anti-diabetic agent that acts by reducing glucose production by the liver and by decreasing intestinal absorption of glucose. It is also believed to improve the insulin sensitivity of tissues elsewhere in the body (increases peripheral glucose uptake and utilization). Metformin improves glucose tolerance in impaired glucose tolerant (IGT) subjects and Type 2 diabetic subjects, lowering both pre- and post-prandial plasma glucose. Metformin is generally not effective in the absence of insulin. Bailey, Diabetes Care 15:755-72 (1992). Metformin (Glucophage™) is commonly administered as metformin HCl. Metformin is also available in an extended release formulation (Glucophage XR™). Dose ranges of metformin are between 10 to 2550 mg per day, and preferably about 250 mg per day systemically. This corresponds to an estimated local dosage of about 200 to about 400 μg/day.

Thiazolidinediones that can be used include troglitazone (Rezulin™), rosiglitazone (sold as Avandia™ by GlaxoSmithKline), pioglitazone (sold as Actos™ by Takeda Pharmaceuticals North America, Inc. and Eli Lilly and Company), ciglitazone, englitazone, and R483 (produced by Roche, Inc.), and rivoglitazone (Sankyo). Such compounds are well-known, e.g., as described in U.S. Pat. Nos. 5,223,522; 5,132,317; 5,120,754; 5,061,717; 4,897,405; 4,873,255; 4,687,777; 4,572,912; 4,287,200; and 5,002,953; and Current Pharmaceutical Design 2:85-101 (1996). The thiazolidinediones work by enhancing insulin sensitivity in both muscle and adipose tissue and to a lesser extent by inhibiting hepatic glucose production. Thiazolidinediones mediate this action by binding and activating peroxisome proliferator-activated receptor-gamma (PPARγ). Effective doses include troglitazone (10-800 mg/day systemically), rosiglitazone (1-20 mg/day systemically, about 6-12 μg/day locally, or about 25-100 μg total drug load on a stent), and pioglitazone (15-45 mg/day systemically, 20-50 μg/day locally, or about 125-300 μg total drug loaded on a stent). Phase II studies with the glitazone, R483, have been completed and show a significant dose-dependent reduction of HbA1c. R483 has been tested at doses of 5-40 mg/day.

Glitazars are non-thiazolidinedione drugs which activate peroxisome proliferator-activated receptor-gamma and -alpha (PPAR-γ and -α). Glitazars that can be used include farglitazar (GlaxoSmithKline), ragaglitazar (Novo Nordisk), KRP-297 (Kyorin/Merck), tesaglitazar (AstraZeneca Galida®), and muraglitazar (Pargluva® Bristol-Myers Squibb). Another example of a drug which acts as a cardioprotectant and reduces ischemic injury (including reperfusion injury) is adenosine. The drug sensitizers which can be administered before or with adenosine to act as adenosine agonists which activate adenosine receptors and protect heart tissue by preconditioning include A(1) receptor, A(2) receptor, or A(3) receptor agonists. These include for example, AMP579 (A(1) and A(2) receptor), dipyridamole (A(1), A(2), and A(3) receptor), N-6-cyclopentyl adenosine (CPA) (A(1) receptor), R(−)-N-6-(2-phenylisopropyl)adenosine (PIA) (A(1) receptor), 2-chloro-N-6-cyclopentyl adenosine (CCPA) (A(1) receptor), ALT 146e (A(2) receptor), Regadenoson (CVT-3146) (A(2) receptor), and N-6-(3-iodobenzyl)adenosine-5′-methyl-carboxamide (A(3) receptor).

D. Other Therapeutic Agents Incorporated into Medical Devices

Other therapeutically active, prophylactic or diagnostic agents can also be incorporated into the device, for delivery primarily murally, luminally, or bi-directionally. The primarily murally delivered agents may include antineoplastics, antimitotics, anti-inflammatories, anti-angiogenics, angiogenic factors, antirestenotics, anti-thrombotics such as heparin, antiproliferatives such as paclitaxel and rapamycin and derivatives thereof.

Other therapeutic agents for use with the present invention may, for example, take the form of small molecules, peptides, lipoproteins, polypeptides, polynucleotides encoding polypeptides, lipids, protein-drugs, protein conjugate drugs, enzymes, oligonucleotides and their derivatives, ribozymes, other genetic material, cells, antisense oligonucleotides, monoclonal antibodies, platelets, prions, viruses, bacteria, eukaryotic cells such as endothelial cells, stem cells, ACE inhibitors, monocyte/macrophages and vascular smooth muscle cells. Such agents can be used alone or in various combinations with one another. For instance, anti-inflammatories may be used in combination with antiproliferatives to mitigate the reaction of tissue to the antiproliferative. The therapeutic agent may also be a pro-drug, which metabolizes into the desired drug when administered to a host. In addition, therapeutic agents may be pre-formulated as microcapsules, microspheres, microbubbles, liposomes, niosomes, emulsions, dispersions or the like before they are incorporated into the matrix. Therapeutic agents may also be radioactive isotopes or agents activated by some other form of energy such as light or ultrasonic energy, or by other circulating molecules that can be systemically administered.

Exemplary classes of therapeutic agents include antiproliferatives, antithrombins (i.e., thrombolytics), immunosuppressants, antilipid agents, anti-inflammatory agents, antineoplastics including antimetabolites, antiplatelets, angiogenic agents, anti-angiogenic agents, vitamins, antimitotics, metalloproteinase inhibitors, NO donors, nitric oxide release stimulators, anti-sclerosing agents, vasoactive agents, endothelial growth factors, beta blockers, AZ blockers, hormones, statins, insulin growth factors, antioxidants, membrane stabilizing agents, calcium antagonists (i.e., calcium channel antagonists), retinoids, anti-macrophage substances, antilymphocytes, cyclooxygenase inhibitors, immunomodulatory agents, angiotensin converting enzyme (ACE) inhibitors, anti-leukocytes, high-density lipoproteins (HDL) and derivatives, cell sensitizers to insulin, prostaglandins and derivatives, anti-TNF compounds, hypertension drugs, protein kinases, antisense oligonucleotides, cardio protectants, petidose inhibitors (increase blycolitic metabolism), endothelin receptor agonists, interleukin-6 antagonists, anti-restenotics, vasodilators, and other miscellaneous compounds.

Antiproliferatives include, without limitation, paclitaxel, actinomycin D, rapamycin, everolimus, ABT-578, tacrolimus, cyclosporin, and pimecrolimus.

Antithrombins include, without limitation, heparin, aspirin, sulfinpyrazone, ticlopidine, ABCIXIMAB, eptifibatide, tirofiban HCL, coumarines, plasminogen, α₂-antiplasmin, streptokinase, urokinase, bivalirudin, tissue plasminogen activator (t-PA), hirudins, hirulogs, argatroban, hydroxychloroquin, BL-3459, pyridinolcarbamate, Angiomax, and dipyridamole.

Immunosuppressants include, without limitation, cyclosporine, rapamycin and tacrolimus (FK-506), ABT-578, everolimus, etoposide, and mitoxantrone.

Antilipid agents include, without limitation, HMG CoA reductase inhibitors, nicotinic acid, probucol, and fibric acid derivatives (e.g., clofibrate, gemfibrozil, gemfibrozil, fenofibrate, ciprofibrate, and bezafibrate).

Anti-inflammatory agents include, without limitation, pimecrolimus, salicylic acid derivatives (e.g., aspirin, insulin, sodium salicylate, choline magnesium trisalicylate, salsalate, dflunisal, salicylsalicylic acid, sulfasalazine, and olsalazine), para-amino phenol derivatives (e.g., acetaminophen), indole and indene acetic acids (e.g., indomethacin, sulindac, and etodolac), heteroaryl acetic acids (e.g., tolmetin, diclofenac, and ketorolac), arylpropionic acids (e.g., ibuprofen, naproxen, flurbiprofen, ketoprofen, fenoprofen, and oxaprozin), anthranilic acids (e.g., mefenamic acid and meclofenamic acid), enolic acids (e.g., piroxicam, tenoxicam, phenylbutazone and oxyphenthatrazone), alkanones (e.g., nabumetone), glucocorticoids (e.g., dexamethaxone, prednisolone, and triamcinolone), pirfenidone, and tranilast.

Antineoplastics include, without limitation, nitrogen mustards (e.g., mechlorethamine, cyclophosphamide, ifosfamide, melphalan, and chlorambucil), methylnitrosoureas (e.g., streptozocin), 2-chloroethylnitrosoureas (e.g., carmustine, lomustine, semustine, and chlorozotocin), alkanesulfonic acids (e.g., busulfan), ethylenimines and methylmelamines (e.g., triethylenemelamine, thiotepa and altretamine), triazines (e.g., dacarbazine), folic acid analogs (e.g., methotrexate), pyrimidine analogs (5-fluorouracil, 5-fluorodeoxyuridine, 5-fluorodeoxyuridine monophosphate, cytosine arabinoside, 5-azacytidine, and 2′,2′-difluorodeoxycytidine), purine analogs (e.g., mercaptopurine, thioguanine, azathioprine, adenosine, pentostatin, cladribine, and erythrohydroxynonyladenine), antimitotic drugs (e.g., vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin), phenoxodiol, etoposide, and platinum coordination complexes (e.g., cisplatin and carboplatin).

Antiplatelets include, without limitation, insulin, dipyridamole, tirofiban, eptifibatide, abciximab, and ticlopidine.

Angiogenic agents include, without limitation, phospholipids, ceramides, cerebrosides, neutral lipids, triglycerides, diglycerides, monoglycerides lecithin, sphingosides, angiotensin fragments, nicotine, pyruvate thiolesters, glycerol-pyruvate esters, dihydroxyacetone-pyruvate esters and monobutyrin.

Anti-angiogenic agents include, without limitation, endostatin, angiostati, fumagillin and ovalicin.

Vitamins include, without limitation, water-soluble vitamins (e.g., thiamin, nicotinic acid, pyridoxine, and ascorbic acid) and fat-soluble vitamins (e.g., retinal, retinoic acid, retinaldehyde, phytonadione, menaqinone, menadione, and alpha tocopherol).

Antimitotics include, without limitation, vinblastine, vincristine, vindesine, vinorelbine, paclitaxel, docetaxel, epipodophyllotoxins, dactinomycin, daunorubicin, doxorubicin, idarubicin, epirubicin, mitoxantrone, bleomycins, plicamycin and mitomycin.

Metalloproteinase inhibitors include, without limitation, TIMP-1, TIMP-2, TIMP-3, and SmaPI.

NO donors include, without limitation, L-arginine, amyl nitrite, glyceryl trinitrate, sodium nitroprusside, molsidomine, diazeniumdiolates, S-nitrosothiols, and mesoionic oxatriazole derivatives.

NO release stimulators include, without limitation, adenosine.

Anti-sclerosing agents include, without limitation, collagenases and halofuginone.

Vasoactive agents include, without limitation, nitric oxide, adenosine, nitroglycerine, sodium nitroprusside, hydralazine, phentolamine, methoxamine, metaraminol, ephedrine, trapadil, dipyridamole, vasoactive intestinal polypeptides (VIP), arginine, and vasopressin.

Endothelial growth factors include, without limitation, VEGF (Vascular Endothelial Growth Factor) including VEGF-121 and VEG-165, FGF (Fibroblast Growth Factor) including FGF-1 and FGF-2, HGF (Hepatocyte Growth Factor), and Ang1 (Angiopoietin 1).

Beta blockers include, without limitation, propranolol, nadolol, timolol, pindolol, labetalol, metoprolol, atenolol, esmolol, and acebutolol.

Hormones include, without limitation, progestin, insulin, the estrogens and estradiols (e.g., estradiol, estradiol valerate, estradiol cypionate, ethinyl estradiol, mestranol, quinestrol, estrond, estrone sulfate, and equilin).

Statins include, without limitation, mevastatin, lovastatin, simvastatin, pravastatin, atorvastatin, and fluvastatin.

Insulin growth factors include, without limitation, IGF-1 and IGF-2.

Antioxidants include, without limitation, vitamin A, carotenoids and vitamin E.

Membrane stabilizing agents include, without limitation, certain beta blockers such as propranolol, acebutolol, labetalol, oxprenolol, pindolol and alprenolol.

Calcium antagonists include, without limitation, amlodipine, bepridil, diltiazem, felodipine, isradipine, nicardipine, nifedipine, nimodipine and verapamil.

Retinoids include, without limitation, all-trans-retinol, all-trans-14-hydroxyretroretinol, all-trans-retinaldehyde, all-trans-retinoic acid, all-trans-3,4-didehydroretinoic acid, 9-cis-retinoic acid, 11-cis-retinal, 13-cis-retinal, and 13-cis-retinoic acid.

Anti-macrophage substances include, without limitation, NO donors.

Anti-leukocytes include, without limitation, 2-CdA, IL-1 inhibitors, anti-CD116/CD18 monoclonal antibodies, monoclonal antibodies to VCAM, monoclonal antibodies to ICAM, and zinc protoporphyrin.

Cyclooxygenase inhibitors include, without limitation, Cox-1 inhibitors and Cox-2 inhibitors (e.g., CELEBREX® and VIOXX®).

Immunomodulatory agents include, without limitation, immunosuppressants (see above) and immunostimulants (e.g., levamisole, isoprinosine, Interferon alpha, and Interleukin-2).

ACE inhibitors include, without limitation, benazepril, captopril, enalapril, fosinopril sodium, lisinopril, quinapril, ramipril, spirapril, and 2B3 ACE inhibitors.

Cell sensitizers to insulin include, without limitation, glitazones, P PAR agonists and metformin.

Antisense oligonucleotides include, without limitation, resten-NG.

Cardio protectants include, without limitation, VIP, pituitary adenylate cyclase-activating peptide (PACAP), apoA-I milano, amlodipine, nicorandil, cilostaxone, and thienopyridine.

Petidose inhibitors include, without limitation, omnipatrilat.

Anti-restenotics include, without limitation, include vincristine, vinblastine, actinomycin, epothilone, paclitaxel, paclitaxel derivatives (e.g., docetaxel), rapamycin, rapamycin derivatives, everolimus, tacrolimus, ABT-578, and pimecrolimus.

PPAR gamma agonists include, without limitation, farglitizar, rosiglitazone, muraglitazar, pioglitazone, troglitazone, and balaglitazone.

Miscellaneous compounds include, without limitation, Adiponectin.

Agents may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods.

Some of the agents described herein may be combined with additives which preserve their activity. For example additives including surfactants, antacids, antioxidants, and detergents may be used to minimize denaturation and aggregation of a protein drug. Anionic, cationic, or nonionic detergents may be used. Examples of nonionic additives include but are not limited to sugars including sorbitol, sucrose, trehalose; dextrans including dextran, carboxy methyl (CM) dextran, diethylamino ethyl (DEAE) dextran; sugar derivatives including D-glucosaminic acid, and D-glucose diethyl mercaptal; synthetic polyethers including polyethylene glycol (PEF and PEO) and polyvinyl pyrrolidone (PVP); carboxylic acids including D-lactic acid, glycolic acid, and propionic acid; detergents with affinity for hydrophobic interfaces including n-dodecyl-β-D-maltoside, n-octyl-β-D-glucoside, PEO-fatty acid esters (e.g. stearate (myrj 59) or oleate), PEO-sorbitan-fatty acid esters (e.g. Tween 80, PEO-20 sorbitan monooleate), sorbitan-fatty acid esters (e.g. SPAN 60, sorbitan monostearate), PEO-glyceryl-fatty acid esters; glyceryl fatty acid esters (e.g. glyceryl monostearate), PEO-hydrocarbon-ethers (e.g. PEO-10 oleyl ether; triton X-100; and Lubrol. Examples of ionic detergents include but are not limited to fatty acid salts including calcium stearate, magnesium stearate, and zinc stearate; phospholipids including lecithin and phosphatidyl choline; CM-PEG; cholic acid; sodium dodecyl sulfate (SDS); docusate (AOT); and taumocholic acid.

Agents for the treatment of ischemic injury may also be delivered using a gene therapy-based approach in combination with an expandable medical device. Gene therapy refers to the delivery of exogenous genes to a cell or tissue, thereby causing target cells to express the exogenous gene product. Genes are typically delivered by either mechanical or vector-mediated methods. Mechanical methods include, but are not limited to, direct DNA microinjection, ballistic DNA-particle delivery, liposome-mediated transfection, and receptor-mediated gene transfer. Vector-mediated delivery typically involves recombinant virus genomes, including but not limited to those of retroviruses, adenoviruses, adeno-associated viruses, herpesviruses, vaccinia viruses, picornaviruses, alphaviruses, and papovaviruses.

E. Additives

Therapeutic agents may be pre-formulated as microcapsules, microspheres, microbubbles, liposomes, niosomes, emulsions, or dispersions prior to incorporation into the delivery matrix.

Any of the pharmaceutically acceptable additives can be combined with the therapeutically active agents prior to or at the time of encapsulation. These may include surfactants, buffering agents, antioxidants, bulking agents, dispersants, pore forming agents, and other standard additives. Surfactants may be used to minimize denaturation and aggregation of a drug, such as insulin. Anionic, cationic, or nonionic surfactants may be used. Examples of nonionic surfactants include but are not limited to sugars including sorbitol, sucrose, trehalose; dextrans including dextran, carboxy methyl (CM) dextran, diethylamino ethyl (DEAE) dextran; sugar derivatives including D-glucosaminic acid and D-glucose diethyl mercaptal; synthetic polyethers including polyethylene glycol (PEG) and polyvinyl pyrrolidone (PVP); carboxylic acids including D-lactic acid, glycolic acid, and propionic acid; detergents with affinity for hydrophobic interfaces including n-dodecyl-.beta.-D-maltoside, n-octyl-.beta.-D-glucoside, PEO-fatty acid esters (e.g. stearate (myrj 59) or oleate), PEO-sorbitan-fatty acid esters (e.g. Tween 80, PEO-20 sorbitan monooleate), sorbitan-fatty acid esters (e.g. SPAN 60, sorbitan monostearate), PEO-glyceryl-fatty acid esters; glyceryl fatty acid esters (e.g. glyceryl monostearate), PEO-hydrocarbon-ethers (e.g. PEO-10 oleyl ether; triton X-100; and Lubrol. Examples of ionic detergents include but are not limited to fatty acid salts including calcium stearate, magnesium stearate, and zinc stearate; phospholipids including lecithin and phosphatidyl choline; CM-PEG; cholic acid; sodium dodecyl sulfate (SDS); docusate (AOT); and taumocholic acid.

### IV. METHODS OF TREATMENT

A. Method of Locally Delivering Drugs to Reduce Ischemic Injury

In one embodiment, one or more drugs which are suited for the reduction of ischemic injury are delivered at or near the site of a reopened occlusion following myocardial infarction or other acute ischemic syndromes. The delivery of the anti-ischemic agent at or near the site of the previous occlusion allows the drugs to be delivered by the blood flow downstream to the reperfused tissue. The drugs can be delivered by a stent containing drugs in openings in the stent as described above. The drugs can also be delivered by a drug coated stent, an implant, microspheres, a catheter, coils, or other local delivery means.

For example, microspheres, coils, lyposomes, or other small drug carriers can be delivered locally at or near the site of a previous occlusion with a catheter or drug delivery stent. These small drug carriers are released and pass downstream into the myocardium where they may implant themselves delivering the drug directly to the ischemic tissue.

The anti-ischemic agent can be released over an administration period which is dependent on the mode of action of the drug delivered. For example, insulin and an insulin sensitizer may be delivered over an administration period of from a few minutes up to weeks. Preferably insulin and the optional insulin sensitizer are delivered over a period of at least 1 hour, more preferably at least 2 hours, and more preferably about 10-72 hours. The insulin and drug sensitizer can be delivered at different times and for different periods. For example, the drug sensitizer may be delivered first and continue through administration of the insulin. The drug sensitizer can be placed in a separate stent or other local drug delivery device for insertion prior to the insulin stent.

In one example, a therapeutic agent for reduction of ischemic injury and an optional drug sensitizer are delivered from a stent primarily in a luminal direction with minimal drug being delivered directly from the stent in the direction of the vessel wall. This stent may be placed alone in the occlusion or may be placed in addition to another stent (bare stent or drug eluting delivery stent) placed in connection with an angioplasty procedure. The stent for delivery of ischemic injury treatment agent(s) may be placed within or adjacent another previously placed stent. The implantation site for the stent may be at or near the site of the occlusion. An implantation site may also be selected at or near a location of a plaque rupture site or a vessel narrowing.

In another example, two anti-ischemic agents for treatment of ischemic injury may be delivered over different administration periods depending on the mode of action of the agents. For example, a fast acting agent may be delivered over a short period of a few minutes while a slower acting agent is delivered over several hours or days.

B. Method of Locally Delivering Drugs to Reduce Ischemic Injury and Inhibit Restenosis

In preferred embodiments, an anti-restenotic agent is delivered primarily from a mural side of a stent to inhibit restenosis in addition to the anti-ischemic agent and/or drug sensitizer, which are delivered primarily from the luminal side of the stent. In one example, the anti-ischemic and/or drug sensitizer are delivered at a first delivery rate for a first administration period, such as over a period of about 1 to about 72 hours, while the anti-restenotic drug is delivered at a second delivery rate for a second administration period, such as over a period of about 3 days or longer, and preferably about 30 days or longer.

Other primarily murally delivered agents include antineoplastics, antiangiogenics, anti-thrombotics, such as heparin, antiproliferatives, such as paclitaxel and Rapamycin and derivatives thereof.

C. Method for Local and Systemic Delivery of Drugs for Reducing Ischemic Injury

In another embodiment, the local delivery of an anti-restenotic agent that does not act on mTOR for reduction of ischemic injury is used in combination with the systemic delivery of an anti-ischemic agent and/or drug sensitizer.

### V. PHARMACEUTICALLY ACCEPTABLE FORMULATIONS

The compounds, or pharmaceutically acceptable salts thereof, including their polymorphic variations, can be formulated with pharmaceutically acceptable carriers. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or an encapsulating material such as liposomes, polyethylene glycol (PEG), PEGylated liposomes, or particles, which is compatible with the other ingredients of the formulation and not injurious to the patient.

The phrases “systemic administration” and “administered systemically” as used herein mean the administration of a compound, drug or other material other than directly into the central nervous system, such that it enters the patient's vascular system.

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

The active compounds (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules, particles, or inlays include, but are not limited to, diluents, binders, lubricants, disintegrants, colorants, stabilizers, and surfactants.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the dosage forms may also contain minor amount of nontoxic auxiliary substances such as wetting or emulsifying agents, dyes, pH buffering agents, or preservatives.

### VI. EXEMPLARY DESCRIPTIONS

A. Insulin and Paclitaxel Stent

A drug delivery stent substantially equivalent to the stent illustrated in FIGS. 2 and 5 having an expanded size of about 3 mm×16 mm is loaded with insulin with a total dosage of about 100-300 micrograms and with paclitaxel with a total dosage of about 10-50 micrograms in the following manner. The stent is positioned on a mandrel and an optional quick degrading base is deposited into the openings in the stent. The quick degrading base is PLGA. A plurality of deposits of insulin and low molecular weight PLGA are then deposited into the openings to form an inlay of drug for the reduction of ischemic injury.

The compositions are deposited in a dropwise manner and are delivered in liquid form by use of a suitable organic solvent, such as DMSO, NMP, or DMAc. A plurality of deposits of insulin and low molecular weight trehalose/PVP matrix are then deposited into the openings to form an inlay of drug for the reduction of ischemic injury. The insulin and polymer matrix are combined and deposited in a manner to achieve an insulin delivery profile which results in essentially 100% released in about 24 to about 72 hours.

The insulin dosage provided on the stent described is about 10-200 micrograms. The dosage has been calculated based on reported studies on systemic infusions of insulin which are estimated to deliver to the heart about 10 micrograms of insulin over a 24 hour period. The total dosage on the stent may range from about 5 micrograms to about 500 micrograms, preferably about 100 to about 400 micrograms. A corresponding total dosage of the insulin sensitizer. Rosiglitazone may range from about 10 to 200 micrograms, preferably about 30 to about 90 micrograms.

A plurality of deposits of high molecular weight PLGA, or other slow degrading polymer, and paclitaxel are deposited over the insulin to provide delivery of the paclitaxel from the cap to the mural side of the stent and the vessel walls. The resorbtion rate of the paclitaxel cap is selected to deliver paclitaxel continuously over an administration period of about 2 or more days.

B. Insulin and Pimecrolimus Stent

A stent for eluting insulin and Pimecrolimus was made substantially according to the description in A above. The stent was loaded with a base region, an insulin drug region, and a Pimecrolimus cap region. The base contained PLGA and/or PEVA; the drug contained insulin 250 μg total drug load (TDL) and PLGA; and the cap contained PEVA, PLGA/PLA-PCL and Pimecrolimus 300 μg TDL, and 6 deposits of PLGA/PLA-PCL.

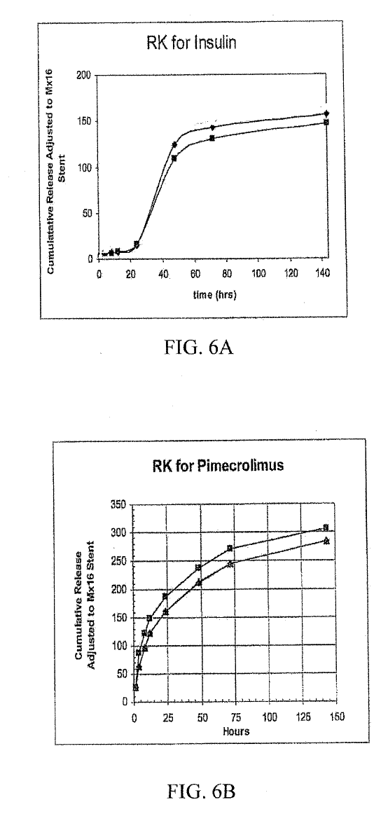

FIGS. 6A and 6B show the release profile of the insulin and Pimecrolimus eluting stent, respectively, over time. As shown in FIG. 6A at least about 50% of the insulin is released in the first 48 hours. As shown in FIG. 6B, the Pimecrolimus is released at a high initial release rate followed by a slower prolonged release.

It is understood that the disclosed methods are not limited to the particular methodology, protocols, and reagents described as these may vary. It is also to be understood that the terminology used herein is for the purpose of describing particular embodiments only, and is not intended to limit the scope of the present invention which will be limited only by the appended claims.

We claim:

1. A method for reducing tissue damage following ischemic injury in a patient, the method comprising:
administering to the patient an anti-ischemic agent which reduces tissue damage due to ischemia and one or more anti-restenotic agent that reduces or prevents restenosis, wherein the anti-ischemic agent and the one or more anti-restenotic agent are administered locally to or near the site of ischemic injury, and wherein the anti-restenotic agent does not reduce the beneficial effects provided by the anti-ischemic agent.

2. The method of claim 1, wherein at least one of the anti-ischemic agent and anti-restenotic agent are administered in a medical device implanted at or near the site of ischemic injury.

3. The method of claim 2, wherein the device is selected from the group consisting of stents, polymeric delivery devices, polymeric particles and polymeric coatings.

4. The method of claim 3, wherein the at least one of anti-ischemic agent and one or more anti-restenotic agent is administered into a blood vessel.

5. The method of claim 4, wherein the at least one of anti-ischemic agent is administered for periods of time sufficient to reduce ischemic injury.

6. The method of claim 4, wherein the anti-restenotic drug is delivered primarily from a mural side of the medical device, and wherein the anti-ischemic agent is delivered primarily from a luminal side of the medical device.

7. The method of claim 1, wherein the anti-restenotic agent and anti-ischemic agent are delivered from an implanted biodegradable polymer.

8. The method of claim 2, wherein the medical device is a stent.

9. The method of claim 1, wherein the anti-ischemic agent is insulin.

10. The method of claim 1, wherein the anti-restenotic agent is selected from the group of compounds consisting of antineoplastics, antimitotics, antiangiogenics, angiogenic factors, anti-thrombotics, antiproliferatives, and anti-inflammatories.

11. The method of claim 1 wherein the anti-restenotic agent is pimecrolimus, sirolimus or paclitaxel.

12. The method of claim 1, wherein the anti-ischemic and anti-restenotic agent are delivered from a polymer.

13. The method of claim 12, wherein the polymer is in the form of polymeric coatings or particles located at or near an occlusion site.

14. The method of claim 12, wherein the anti-ischemic agent, anti-restenotic agent and a biocompatible polymer matrix are deposited within openings in an implantable medical device for local delivery to an occlusion site.

15. The method of claim 12, wherein the anti-ischemic agent, anti-restenotic agent and a biocompatible polymer are deposited within openings in an implantable medical device and wherein a barrier region is provided which substantially prevents delivery of the anti-ischemic agent to the artery wall.

16. The method of claim 14, wherein the anti-ischemic agent is delivered over a period of about 1 to 72 hours.

17. The method of claim 16, wherein the anti-restenotic agent is delivered over a period of about 30 days or longer.

18. The method of claim 17, wherein the anti-ischemic agent and the anti-restenotic agent are delivered at different rates.

19. An implantable stent for reducing tissue damage following ischemic injury in a patient, comprising:
an expandable stent structure:
an anti-ischemic agent affixed to the stent structure, wherein the anti-ischemic agent reduces tissue damage due to ischemia; and
one or more anti-restenotic agent that reduces or prevents restenosis wherein the anti-restenotic agent does not reduce the beneficial effects provided by the anti-ischemic agent.

20. The stent of claim 19, wherein the anti-ischemic agent and anti-restenotic agent are released for at least one hour.

21. The stent of claim 19, wherein the anti-ischemic agent is released for about 10 to about 48 hours.

22. The stent of claim 19, wherein the anti-ischemic agent is insulin and the therapeutic dosage is about 5 to about 800 micrograms.

23. The stent of claim 22, wherein the insulin is affixed to the stent by depositing in holes in the stent.

24. The stent of claim 19, wherein the stent further comprises one or more drug sensitizers.

25. The stent of claim 24, wherein the drug sensitizer is an insulin sensitizer.

26. The stent of claim 25, wherein the insulin sensitizer is selected from the group consisting of biguanides, thiazolidinediones, and glitazars.

27. The stent of claim 23, wherein the anti-restenotic agent is affixed to the stent by depositing in holes in the stent.

28. The stent of claim 19, wherein the anti-restenotic agent and anti-ischemic agent are delivered from an implanted biodegradable polymer.

29. The method of claim 19, wherein the anti-ischemic agent is delivered over a period of about 1 to 72 hours.

30. The method of claim 29, wherein the anti-restenotic agent is delivered over a period of about 30 days or longer.
